(19) United States
(12) Patent Application Publication
(10) Pub. No.: US 2021/0299375 A1
(43) Pub. Date: Sep. 30, 2021

(54) VENTILATORS AND METHODS FOR STABILIZING VALVE POSITION IN VENTILATORS

(71) Applicant: Covidien LP, Mansfield, MA (US)

(72) Inventor: Julio Jenaro, Galway (IE)

(73) Assignee: Covidien LP, Mansfield, MA (US)

(21) Appl. No.: 17/171,823

(22) Filed: Feb. 9, 2021

Related U.S. Application Data
(60) Provisional application No. 63/000,399, filed on Mar. 26, 2020.

(51) Int. Cl. A61M 16/00; A61M 16/20

(52) U.S. Cl. CPC A61M 16/024; A61M 16/205; A61M 16/0051; A61M 2205/581; A61M 2205/18; A61M 2205/3334; A61M 2205/583; A61M 2016/0027

(57) ABSTRACT

Ventilators and methods of stabilizing valve position on a ventilator are provided. The method includes controlling fluid flow through a valve with a first control loop. Determining that the valve is in a steady state condition with respect to the first control loop. Activating a second control loop once the steady state condition is determined. Controlling a position of the valve with the second control loop based on a position sensor that measures linear displacement of the valve, and disabling the second control loop when the first control loop is in operation. The first control loop is independent from the second control loop, and the second control loop operates at a smaller sampling interval period than the first control loop.

### CROSS-REFERENCE TO RELATED APPLICATIONS

This application claims priority to and the benefit of the filing date of provisional U.S. Patent Application No. 63/000,399, filed Mar. 26, 2020, the disclosure of which is hereby incorporated herein by reference.

### INTRODUCTION

Medical ventilator systems are used to provide ventilatory and supplemental oxygen support to patients. These ventilators typically include a connection for pressurized gas (air, oxygen) that is delivered to the patient through a conduit or tubing. Additionally, fluid control valves are provided so as to control flow rate and/or pressure of the gas through the conduit or tubing. These control valves can be adjustable so as to react to changes in flow rate and/or pressure of the gas and patient requirements. However, ventilators are also moveable, for example, configured to move with the patient during patient transport, and thus, are subject to forces that induce vibration in the ventilators. These vibrations can cause undesirable movement of the control valves that affects the flow rate and/or pressure of the gas through the conduit or tubing.

It is with respect to this general technical environment that aspects of the present technology disclosed herein have been contemplated. Furthermore, although a general environment has been discussed, it should be understood that the examples described herein should not be limited to the general environment identified herein.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Among other things, aspects of the present disclosure include systems and methods for stabilizing valve position on a ventilator. In an aspect, the technology relates to a method of stabilizing valve position on a ventilator, the method including: controlling fluid flow through a valve with a first control loop; determining that the valve is in a steady state condition with respect to the first control loop; activating a second control loop once the steady state condition is determined; controlling a position of the valve with the second control loop based on a position sensor that measures linear displacement of the valve; and disabling the second control loop when the first control loop is in operation, wherein the first control loop is independent from the second control loop, and wherein the second control loop operates at a smaller sampling interval period than the first control loop.

In an example, controlling fluid flow through the valve with the first control loop includes setting a flow position of the valve based on a target fluid flow rate or pressure. In another example, the method includes after determining that the valve is in a steady state condition, storing a steady state position of the valve. In yet another example, controlling the position of the valve with the second control loop is based at least partially on the stored steady state position of the valve relative to the measured linear displacement of the valve. In still another example, determining that the valve is in a steady state condition includes: measuring linear displacement of the valve over a plurality of sampling interval periods; and verifying that the position of the valve remains substantially consistent over two or more sampling interval periods of the plurality of sampling interval periods. In an example, the method further includes generating an alarm on the ventilator when the second control loop is active. In another example, controlling the position of the valve is performed by electric current channeled to the valve.

In another aspect, the technology relates to a ventilator including: a valve configured to regulate fluid flow therethrough; a valve position sensor; a processor; and a memory storing computer executable instruction that when executed by the processor cause the ventilator to perform a set of operations that stabilizes the position of the valve including: controlling the fluid flow through the valve with a first control loop; determining that the valve is in a steady state condition with respect to the first control loop; activating a second control loop once the steady state condition is determined; controlling a position of the valve with the second control loop based on the valve position sensor that measures linear displacement of the valve; and disabling the second control loop when the first control loop is in operation, wherein the first control loop is independent from the second control loop, and wherein the second control loop operates at a smaller sampling interval period than the first control loop.

In an example, the operation of controlling the fluid flow through the valve with the first control loop includes setting a flow position of the valve based on a target fluid flow rate or pressure. In another example, the set of operations further include after determining that the valve is in a steady state condition, storing a steady state position of the valve. In yet another example, the operation of controlling the position of the valve with the second control loop is based at least partially on the stored steady state position of the valve relative to the measured linear displacement of the valve. In still another example, the operation of determining that the valve is in a steady state condition includes: measuring linear displacement of the valve over a plurality of sampling interval periods; and verifying that the position of the valve remains substantially consistent over two or more sampling interval periods of the plurality of sampling interval periods. In an example, the ventilator further includes an audio, a visual, or an audio and visual alarm, and the set of operations further includes generating an alarm on the ventilator when the second control loop is active. In another example, the operation of controlling the position of the valve is performed by electric current channeled to the valve.

In another aspect, the technology relates to a ventilator including: a fluid flow circuit; an exhalation valve coupled in fluidic communication with the fluid flow circuit and configured to at least partially control fluid flow through the fluid flow circuit; a first sensor coupled to the fluid flow circuit and configured to measure flow rate, pressure, or flow rate and pressure of the fluid flow through the fluid flow circuit; a second sensor coupled to the exhalation valve and configured to measure a position of the exhalation valve; and a controller including a processor and memory coupled in communication with the exhalation valve, the first sensor, and the second sensor, wherein the controller is configured to control a position of the exhalation valve based on a target fluid flow rate or pressure in a first control loop having the first sensor and control the position of the exhalation valve based on measured linear displacement from the second sensor in a second control loop, wherein the first control loop is independent from the second control loop, and wherein the second control loop operates at a smaller sampling interval period than the first control loop.

In an example, the controller drives position of the exhalation valve by electric current. In another example, the electric current is based at least partially on voltage calculated by the controller. In yet another example, during operation of the first control loop, the second control loop is disabled by the controller. In still another example, the ventilator further includes an audio, a visual, or an audio and visual alarm. In an example, the ventilator further includes at least one dampener configured to at least partially isolate the exhalation valve from vibratory forces.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a schematic diagram illustrating an exemplary ventilator connected to a human patient.

FIG. 2 is a block-diagram illustrating the ventilator shown in FIG. 1.

FIG. 3 is a block-diagram of an exhalation module of the ventilator shown in FIG. 1.

FIG. 4 is another block-diagram of the exhalation module shown in FIG. 3.

FIG. 5 is a flowchart illustrating a method for stabilizing valve position on a ventilator.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. Ventilators may provide pressure regulating valves (or regulators) connected to sources of pressurized air and pressurized oxygen. The regulating valves function to regulate flow rate and/or pressure so that respiratory gases having a desired concentration are supplied to the patient at required or desired flow rates and pressures. However, medical ventilators can also be movable, for example, remain attached to the patient while the patient is being transported. In other examples, medical ventilators may be transported in vehicles, such as ambulances or air ambulances (e.g., helicopters), while providing breathing support to the patient. During any type of transportation, the ventilators may be subject to vibratory forces.

The methods and systems described herein enable for valves within the ventilator to be stabilized during vibratory events so that the required or desired fluid pressures and flow rates are maintained during ventilator operation. In an example, the valve position control has two positioning control loops. A primary loop has a sensor and controller that positions the ventilator valve at a flow position corresponding to the required or desired fluid pressure and flow rate. A stabilization loop is also included and has a valve position sensor and controller that operates at a faster sampling period rate. Based on the stabilization loop, the control of the valve position can be quickly adjusted so as to account for the vibratory forces acting on the ventilator. This stabilization control increases performance of the ventilator and can reduce or prevent undesirable fluid pressure and flow rate changes during ventilation. With these concepts in mind, several exemplary methods and systems are discussed below.

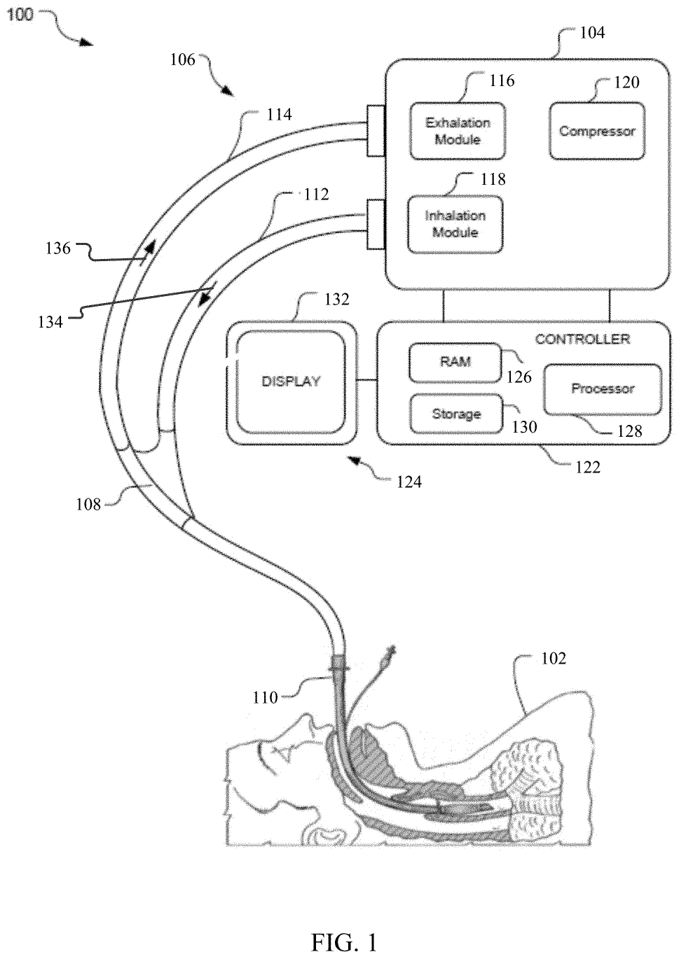

FIG. 1 is a schematic diagram illustrating an exemplary ventilator 100 connected to a human patient 102. The ventilator 100 includes a pneumatic system 104 (also referred to as a pressure generating system 104) for circulating breathing gases to and from the patient 102 via a ventilation tubing system 106 (also referred to as a fluid flow circuit 106), which couples the patient to the pneumatic system 104 via an invasive (e.g., endotracheal tube, as shown) or a non-invasive (e.g., nasal mask) patient interface.

The ventilation tubing system 106 may be a two-limb (shown) or a one-limb circuit for carrying gases to and from the patient 102. In a two-limb example, a fitting, typically referred to as a “wye-fitting” 108, may be provided to couple a patient interface 110 to an inhalation limb 112 and an exhalation limb 114 of the ventilation tubing system 106.

The pneumatic system 104 may have a variety of configurations. In the present example, the system 104 includes an exhalation module 116 coupled with the exhalation limb 114 and an inhalation module 118 coupled with the inhalation limb 112. A compressor 120 or other source(s) of pressurized gases (e.g., air, oxygen, and/or nitrogen) is coupled in flow communication with inhalation module 118 to provide a gas source for ventilatory support via the inhalation limb 112. The pneumatic system 104 may include a variety of other components, including mixing modules, valves, sensors, tubing, accumulators, filters, etc.

A controller 122 is operatively coupled with pneumatic system 104 and an operator interface 124 that may enable a technologist to interact with the ventilator 100 (e.g., change ventilator settings, select operational modes, view monitored parameters, etc.). The controller 122 may include memory 126, one or more processors 128, storage 130, and/or other components of the type found in command and control computing devices. For example, the processor 128 can be a processor, a complex programmable logic device “CPLD,” a field programmable gate array “FPGA,” or a digital signal processor “DSP.” In the example, the operator interface 124 includes a display 132 that may be touch-sensitive and/or voice-activated, enabling the display 132 to serve both as an input and output device.

The memory 126 includes non-transitory, computer-readable storage media that stores software that is executed by the processor 128, and which controls the operation of the ventilator 100. In an example, the memory 126 includes one or more solid-state storage devices such as flash memory chips. In an alternative example, the memory 126 may be mass storage connected to the processor 128 through a mass storage controller (not shown) and a communications bus (not shown). Although the description of computer-readable media contained herein refers to a solid-state storage, it should be appreciated by those skilled in the art that computer-readable storage media can be any available media accessed by the processor 128. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer device. Communication between components of the ventilator 100 or between the ventilator 100 and other therapeutic equipment and/or remote monitoring systems may be conducted via wired or wireless means as required or desired.

The ventilator 100 may engage one or more data collection sensors (not shown) to monitor various parameters that may be measured or calculated based on the system between the ventilator 100 and the patient 102. For example, the data collection sensors may collect one or more of gas flow, pressure, volume, or any other measurement that may be measured, calculated, or derived based on ventilation of the patient 102, measured at both the inhalation module 118 and exhalation module 116 of the ventilator. While measuring and collecting data, the ventilator 100 may analyze, graph, or perform other calculations to determine other desired parameters, such as a exhalation flow rate and/or pressure at the exhalation module 116. This measured, collected, or calculated data may be used by the technologist or ventilator 100 when determining potential adjustments or changes to settings of the ventilator 100 in order to optimize patient-ventilator interaction.

In operation, the inhalation module 118 is configured to deliver gases (e.g., a flow of fluid) 134 to the patient 102 and the exhalation module 116 is configured to receive gases (e.g., the flow of fluid) 136 from the patient. In aspects, the operation of the ventilator 100 may be based at least partially on flow rate of the exhalation flow 136 and/or pressure of the exhalation flow 136.

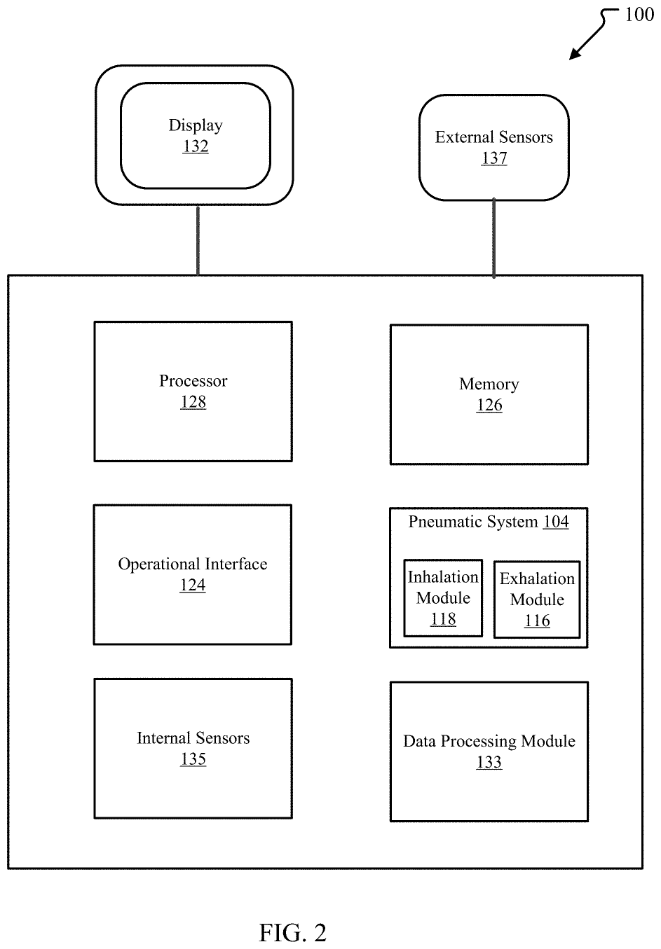

FIG. 2 is a block-diagram illustrating the ventilator shown in FIG. 1 with its various operational modules and components. That is, ventilator 100 may include, among other things, the memory 126, one or more processors 128, the operator interface 124, and the pneumatic system 104 (which may further include the inhalation module 118 and the exhalation module 116). The processors 128 may be configured with a clock or other time keeping instrument whereby elapsed time may be monitored by the ventilator 100 and certain operations can be performed at predetermined time sampling periods, intervals, or cycles.

The ventilator 100 may also include the display 132. The display 132 can be integral with the ventilator 100 or a discrete device that is communicatively coupled to ventilator 100. The display 132 provides various input screens, for receiving input from the technologist, and various display screens, for presenting useful information to the technologist. The display 132 is configured to communicate with the operator interface 124 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, alarms, patient information, parameter settings, etc.). The elements may include controls, graphics, charts, tool bars, input fields, icons, etc. Alternatively, other suitable means of communication with the ventilator 100 may be provided, for instance by a wheel, keyboard, mouse, or other suitable interactive device. Thus, the operator interface 124 may accept commands and input through the display 132 as required or desired. The display 132 may also provide information in the form of various ventilatory data regarding the physical condition of a patient and/or a prescribed respiratory treatment. The information may be data that is input into the system, based on data collected by the ventilator 100 and one or more internal 135 or external 137 sensors, derived from data by a data processing module 133, and the useful information may be displayed to the clinician in the form of graphs, wave representations (e.g., a waveform), pie graphs, numbers, or other suitable forms of graphic display. For example, the data processing module 133 may be operative to determine valve position information that stabilize one or more valves of the ventilator and/or display information regarding the ventilator valves, as detailed herein.

The pneumatic systems 104 may oversee ventilation of a patient according to ventilatory settings. Ventilatory settings may include any appropriate input for configuring the ventilator to deliver breathable gases to a particular patient, including measurements and settings associated with exhalation flow rate and/or pressure of the breathing circuit. Ventilatory settings may be entered by a technologist, e.g., based on a prescribed or target treatment protocol for the particular patient, or automatically generated by the ventilator, e.g., based on attributes (i.e., age, diagnosis, ideal body weight, gender, etc.) of the particular patient according to any appropriate standard protocol or otherwise. In some cases, certain ventilatory settings may be adjusted based on the exhalation flow rate and/or pressure, e.g., to optimize the prescribed treatment. Ventilatory settings may include inhalation flow rate and/or pressure, frequency of delivered breaths (e.g., respiratory rate), tidal volume, positive end-expiratory pressure “PEEP,” etc.

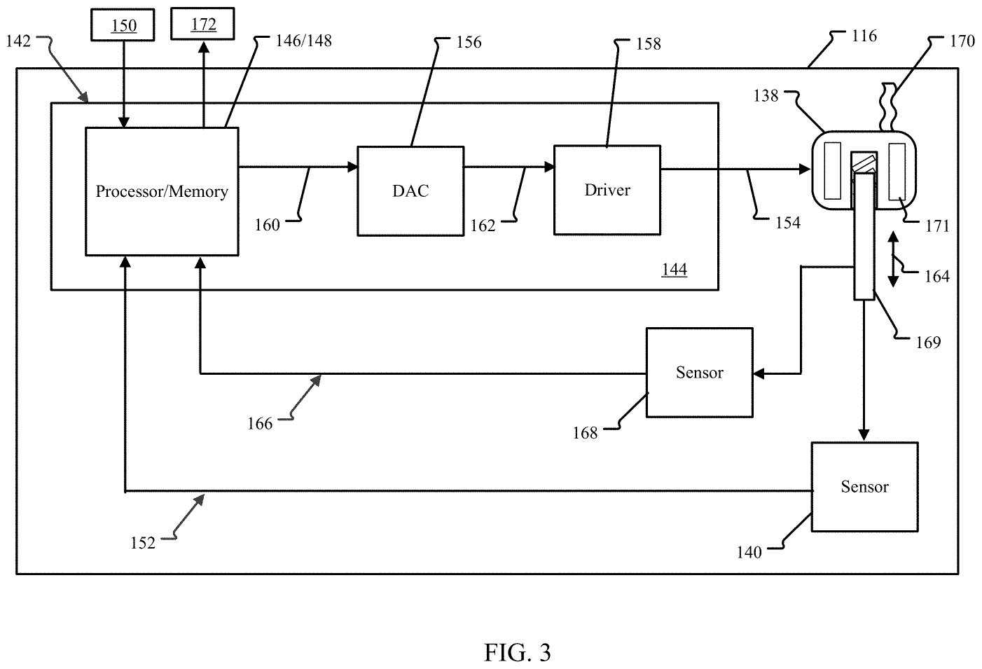

FIG. 3 is a block-diagram of the exhalation module 116 of the ventilator 100 (shown in FIG. 1). The exhalation module 116 includes an exhalation valve 138 coupled in fluidic communication with the fluid flow circuit 106 (shown in FIG. 1) and is configured to regulate a flow of fluid through the circuit 106. For example, the exhalation valve 138 may be used to regulate pressure of the fluid flow through the circuit 106, and a more open position of the valve 138 may correspond to a lower exhalation pressure, while a more closed position of the valve 138 may correspond to a higher exhalation pressure. In another example, the exhalation valve 138 may be used to regulate flow rate of the fluid flow through the circuit 106. It should be appreciated that the exhalation pressure and/or flow rate may additionally or alternatively be based on other flow variables, including air breathed into the circuit 106 by the patient, a setting of an inhalation flow valve (not shown), resistance of the circuit 106, etc. In the example, the exhalation valve 138 is an electromagnetically controlled valve (e.g., a solenoid valve) so that valve position can be adjusted via electric current channeled to a valve coil, and thereby regulating fluid flow therethrough. It should be appreciated, that the exhalation valve 138 may be any other type of flow control valve, (e.g., pneumatic, motor, etc.) that enables the ventilator 100 to function as described herein.

The fluid flow through the exhalation valve 138 is measured by a sensor 140. The sensor 140 may be any sensor or combination of sensors that enables measurement of flow rate, pressure, or flow rate and pressure of the exhalation fluid flow. For example, a thermistor, a hot-wire anemometer, a hot plate, cold wire, vane sensor, etc., and may have a variety of components to otherwise ensure accuracy of the measurements.

The exhalation module 116 also includes a controller 142 operably coupled in communication with the exhalation valve 138 and coupled in communication with the sensor 140. The controller 142 may include a printed circuit board assembly 144 having memory 146 and a processor 148. In an aspect, the processor 128 can be a processor, a complex programmable logic device “CPLD,” a field programmable gate array “FPGA,” or a digital signal processor “DSP.” In some examples, the controller 142 may be part of the pneumatic system controller 122 (shown in FIG. 1), and both the exhalation module 116 and the inhalation module 118 (shown in FIG. 1) may be controlled by the same components within the ventilator 100. In other examples, controllers for the exhalation module 116 and the inhalation module 118 may be discrete components that are coupled in communication together.

In operation, the controller 142 is configured to receive at least one parameter value 150 that is associated with the target flow rate and/or pressure of fluid through the flow circuit 106. The parameter value 150 can be based on inputted, measured, collected, or calculated data from the ventilator 100 to optimize patient-ventilator interaction. Based on the required or desired fluid flow rate and/or pressure, the controller 142 selectively positions the exhalation valve 138 at a flow position so that the flow circuit 106 can achieve the target fluid flow rate and/or pressure. The sensor 140 measures flow rate and/or pressure through the exhalation valve 138 and can provide a first control loop 152 so as to provide feedback and adjust the flow position of the exhalation valve 138 and match the target fluid flow rate and/or pressure. In the example, positional control of the exhalation valve 138 is provided by electrical current 154 (e.g., generating a positioning force to the valve) produced by the controller 142. In an aspect, the controller 142 also includes a digital-to-analog converter (DAC) 156 and a voltage-to-current driver 158. As such, based on the received flow parameter value 150, the processor 148 can calculate a digital current value 160 that is provided to the DAC 156. The DAC 156 generates a voltage 162 based on the digital current value 160, which is then converted to the electrical current 154 at the driver 158 so as to provide a force, via the current 154, to the exhalation valve 138 and selectively position the exhalation valve 138. Then based on the measured flow rate and/or pressure from the sensor 140, the digital current value 160 can be updated so as to reposition the exhalation valve 138 and achieve the target fluid flow rate and/or pressure in the first control loop 152. It should be appreciated that any other method and/or system to generate the required or desired electrical current 154 can be used as required or desired.

In the example, however, when vibratory forces 164 act on the ventilator 100, the position of the exhalation valve 138 may change, which undesirable affects the fluid flow rate and/or pressure in the flow circuit 106. These vibratory forces 164 can act in a direction that is substantially parallel to the exhalation valve's axis, and for example, can induce flow rate and/or pressure changes in the flow circuit 106 of up to 30% or more. Accordingly, in the examples described herein, a second control loop 166 is coupled to the exhalation valve 138 so as to stabilize position of the valve 138 when vibratory forces 164 are present and during operation of the first control loop 152. In an aspect, the first control loop 152 operates at a different and slower sampling period than the second control loop 166. As such, the exhalation valve 138 can still be used to regulate fluid flow rate and/or pressure in the flow circuit 106 and be adjusted accordingly, while also being stabilized against undesirable vibratory forces 164, which often necessitate a faster control loop and sampling period so as to generate the positional control of the valve 138.

The exhalation module 116 also includes a valve position sensor 168 coupled to the exhalation valve 138 and in communication with the controller 142. The valve position sensor 168 is configured to measure linear displacement of the valve 138. For example, where the valve 138 is a solenoid valve, as depicted in FIG. 3, the valve position sensor 168 may measure the linear displacement of a piston 169 of the exhalation valve 138. As will be appreciated by those having skill in the art, a solenoid valve generally operates by moving the piston 169 within a cavity. In some examples, the piston 169 may be at least partially biased within the cavity by a spring. The piston 169 may be moved by providing an electric current through one or more coils 171. The current through the coils 171 generates a magnetic force that moves the piston 169. Thus, the amount of current provided affects the position of the piston 169. The position of the piston 169 affects the amount of fluid that can pass through the valve 138, and thus affects the flow rate and pressure of breathing gases in a patient circuit. In an aspect, the valve position sensor 168 may be a linear variable differential transducer (LVDT). In other examples, the valve position sensor 168 may be any other type of linear displacement sensor that allows the ventilator to function as described herein. For example, encoders, resistive sensors, capacitive sensors, inductive sensors (e.g., LVDT, linear variable reluctance transducers (LVRT), or linear variable inductance transducers (LVIT)), optical sensors, magnetic sensors, time-of-flight sensors, and the like.

Based on the displacement measurements from the sensor 168, the controller 142 can apply a stabilizing force (e.g., a change of the electrical current 154) to the exhalation valve 138 so as to reduce or prevent movement of the exhalation valve 138 induced by the vibratory forces 164. The stabilizing force may be used to effectively counteract the vibratory forces 164. For example, the stabilizing force can be applied by the second control loop 166 to update and revise the digital current value 160 that is used to position the exhalation valve 138. In the example, the first control loop 152 is independent from the second control loop 166 so that the second control loop 166 can be disabled by the controller 142 when the exhalation valve 138 is being positioned for fluid flow rate and/or pressure regulation. In the example, the second control loop 166 can be used to increase the electrical current 154 channeled to the exhalation valve 138 so as to maintain position of the valve during vibration events and overcome the vibratory forces 164, or adjust the electrical current 154 channeled to the exhalation valve so as to reposition the valve 138 after movement caused by the vibration events.

Additionally or alternatively, the exhalation valve 138 may be coupled to one or more dampeners 170 that are configured to absorb the vibratory forces 164 and isolate the exhalation valve 138 from movement by the vibratory forces 164. The dampeners 170 can be coupled to the exhalation valve 138 itself, or be used system wide, for example, to isolate the entire pneumatic system 104 (shown in FIG. 1) or to isolate the entire ventilator 100 (shown in FIG. 1) from the vibratory forces 164. The dampener 170 can be mechanical (e.g., springs or elastomeric pads), hydraulic, pneumatic, and/or electromagnetic. These dampeners 170 can be active systems or passive systems as required or desired.

The controller 142 may also be coupled to an alarm 172. The alarm 172 may be an audio, a visual, or an audio and visual alarm that can alert the technologist to when high vibratory forces 164 are present on the ventilator 100. In an aspect, the alarm 172 can be displayed on the display 132 (shown in FIG. 1). In an aspect, when the second control loop 166 is in operation, the alarm 172 may be triggered so that the technologist can be aware of external forces acting on the ventilator 100. In another aspect, during high vibratory force events the alarm 172 may be triggered so that the technologist is aware and can perform remedial procedures. In yet another aspect, the alarm 172 may be triggered in stages as required or desired.

It should be appreciated that while the second control loop 166 illustrated in FIG. 3 is part of the control system of the exhalation valve 138, this stabilizing valve control can be used with any other electromagnetic valve within the ventilator 100. For example, the inhalation module 118 (shown in FIG. 1) can additionally or alternatively have a flow control valve that is stabilized during vibration events as described herein. Additionally or alternatively, when the second control loop 166 is disabled, the position sensor 168 may be used with the first control loop 152 so as to provide a more accurate reading of the position of the piston 169 that can then be extrapolated into a more accurate reading of fluid flow rate or pressure for fluid flow regulation.

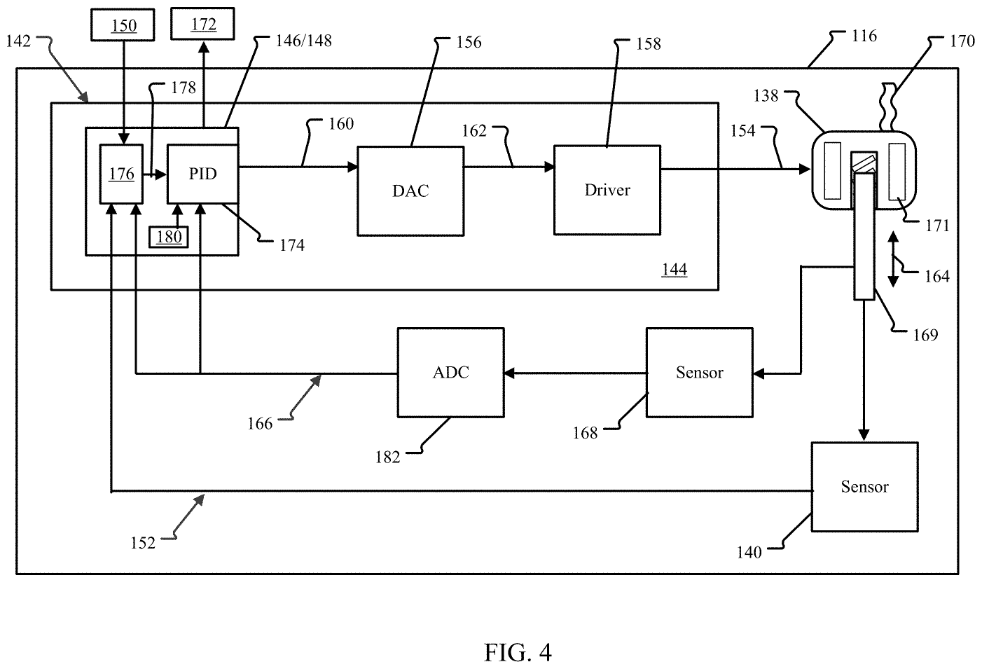

FIG. 4 is another block-diagram of the exhalation module 116 of the ventilator 100 (shown in FIG. 1). Certain components are described above, and thus, are not necessarily described further. As illustrated in FIG. 4, further components of the exhalation module 116 are described that enable operation the exhalation valve 138. The controller 142 includes a control loop mechanism 174 that employs control from the valve position sensor 168 so as to provide continuously modulated control of the exhalation valve 138 based on the applied vibratory forces 164. In the example, the control loop mechanism 174 is a proportional-integral-derivative (PID) controller, however, it is appreciated that any other adaptive controller may be used as required or desired (e.g., fuzzy logic, fuzzy logic PID controller, or the like). In operation, the control loop mechanism 174 automatically applies accurate and responsive correction to the position of the exhalation valve 138 so as to maintain fluid flow rate and/or pressure within the flow circuit 106 (shown in FIG. 1) that experience vibratory forces 164. The control loop mechanism 174 can also be selectively activated and disengaged so that the exhalation valve 138 can be positioned and/or repositioned to control the flow rate and/or pressure of the fluid within the circuit as required or desired. The controller 142 also includes a main control algorithm 176 configured to receive the parameter value 150 and position the exhalation valve 138 at the target flow rate and/or pressure.

In operation, the exhalation module 116 first positions the exhalation valve 138 at a flow position that is associated with a target fluid flow rate and/or pressure of fluid through the valve 138. More specifically, the controller 142 receives at least one parameter value 150 that is associated with the target flow rate and/or pressure of fluid through the flow circuit 106. In an example, the parameter value 150 is associated with the pressure of the fluid flow or a flow rate of the fluid flow. The parameter value 150 may be a flow rate or a pressure value target entered by a technologist, e.g., based on a prescribed treatment protocol for the particular patient, or automatically generated (e.g., calculated) by the ventilator, e.g., based on attributes (i.e., age, diagnosis, ideal body weight, gender, etc.) of the particular patient according to any appropriate standard protocol or otherwise. In other cases, the parameter value 150 may be adjusted based on the exhalation flow and/or pressure measurements, inhalation flow and/or pressure measurements, e.g., to optimize the prescribed treatment. Measurements can be direct measurements or calculated averages. In still other examples, the parameter value 150 can reference valve position, current applied, breath frequency, PEEP, or any other indicator that can be associated with fluid flows and/or pressure.

Based on the parameter value 150, the main control algorithm 176 determines a first digital current value 178 that is used to position the exhalation valve 138 towards the flow position. In some examples, a look-up table may be used to calculate the first digital current value 178 from the parameter value 150. In other examples, the processor 148 may apply an algorithm to calculate the first digital current value 178 from the parameter value 150. Because setting the flow position of the exhalation valve 138 is part of the first control loop 152, the control loop mechanism 174 is disabled so that the first digital current value 178 is used as the digital current value 160 that is then used to position the exhalation valve 138 towards the flow position that will allow for the target fluid flow rate and/or pressure.

As described above, the digital current value 160 is utilized to determine and generate the current 154 that physically positions the exhalation valve 138. In the example, when the digital current value 160 is based on the first digital current value 178, the controller 142 positions the exhalation valve 138 at the flow position that enables the required or desired fluid properties to be achieved. For example, the amount of current that is applied to the valve 138 controls the position of the valve. Thus, the current 154 provided to the exhalation valve 138 generates a magnetic force to properly position the valve. As one example, the exhalation valve 138 may be a solenoid valve. Increasing or decreasing the current 154 through a coil of the solenoid valve causes the plunger or piston to move within an orifice. The position of the piston may control the size of the airway, thus allowing for more or less flow rate of fluid and/or more or less pressure.

The sensor 140 measures the fluid flow through the exhalation valve 138 and provides feedback for the first control loop 152 to adjust the flow position of the exhalation valve 138 and regulate the fluid flow to achieve the target flow rate and/or pressure of the fluid. As such, the first control loop 152, via the main control algorithm 176, is used to modify the first digital current value 178 (that then gets passed through the control loop mechanism 174) as required or desired. This first control loop 152 operates at a larger time sampling period than the second control loop 166. In an aspect, the measurement time sampling period of the first control loop 152 is about 50 to 100 times more than the measurement time sampling period of the second control loop 166. As such, the first control loop 152 is slower than the second control loop 166.

Once the exhalation valve 138 is positioned via the first control loop 152, the controller 142, via the valve position sensor 168, then monitors the exhalation valve 138 to determine when a steady state condition is achieved. The steady state condition relates to the position of the exhalation valve 138 remaining unchanged, or relatively unchanged within a degree of error, over a period of time, and thus, at a steady state position. In an aspect, the degree of error can be within ±5% of overall valve displacement length. In another aspect, the degree of error can be within ±1% of overall valve displacement length. For example, a steady state condition may be determined by measuring valve displacement over a plurality of sampling periods, then verifying that a position of the exhalation valve 138 remains substantially consistent over two or more of the sampling periods. This position of the exhalation valve 138 then corresponds to a steady state position of the steady state condition. Verifying consistency of the position of the valve can include the valve maintaining its position over a plurality of measurement cycles that correspond to substantially no positional change of the exhalation valve 138 (e.g., 2, 3, or 4 cycles that measure the valve at approximately the same position). In another example, verifying consistency of the position of the valve can include the valve averaging displacement measurements over a plurality of measurement cycles that correspond to substantially no positional change of the exhalation valve 138 (e.g., 2, 3, or 4 cycles that have an average measurement that is approximately at the same position).

The steady state position of the exhalation valve 138 in the steady state condition can be stored at a data store location 180 within the memory for further use regarding stabilizing the valve as described herein. In an aspect, the steady state position of the exhalation valve 138 is utilized in the second control loop 166 and in the control loop mechanism 174. This is because the second control loop 166 is disengaged during operation of the first control loop 152. Operation of the second control loop 166 is described further below.

When a steady state condition is determined, the controller 142 can activate the second control loop 166 to stabilize the exhalation valve 138 in the steady state position. In the second control loop 166, the valve position sensor 168 measures movement (e.g., linear displacement) of the exhalation valve 138 from the steady state position towards a second or displaced position. This measurement can be an analog measurement that is converted to a digital value at an analog-to-digital converter (ADC) 182 prior to being received at the controller 142. In an example, measuring movement of the exhalation valve 138 can include measuring the overall displacement of the exhalation valve 138. In another example, measuring movement of the exhalation valve 138 can include measuring relative movement and relating the movement back to the steady state position. Based on the measured displacement of the exhalation valve 138 (e.g., from the vibratory forces 164), the control loop mechanism 174 compares the measured displacement of the exhalation valve 138 to the stored steady state position at the data store location 180. This comparison then determines a second digital current value (not illustrated) that is utilized to keep the exhalation valve 138 at the stored steady state position.

In the second control loop 166, the control loop mechanism 174 uses the second digital current value as the digital current value 160 that is provided to determine and generate the current 154 that physically positions the exhalation valve 138 relative to the steady state position. In the example, when the digital current value 160 is based on the second digital current value, the controller 142 stabilizes the exhalation valve 138 at the steady state position based at least partially on the vibratory forces 164 within the system. In the example, the second digital current value is different than the first digital current value because of the vibratory forces 164. For example, the amount of current that is applied to the valve 138 is based on the vibratory forces 164 so as to stabilize the position of the valve. Thus, the current 154 provided to the exhalation valve 138 generates a magnetic force to properly stabilize the valve and counteract the vibratory forces 164, and the magnetic force moves a piston within the exhalation valve 138. Accordingly, pressure changes through the fluid flow circuit 106 (shown in FIG. 1) based on valve position changes from vibratory forces 164 are reduced or prevented. Because the control loop mechanism 174 is constantly receiving measurements from the position sensor 168, the second digital current value and the resulting current 154 are consistently being updated and revised, and changing vibratory forces can be accounted for.

The second control loop 166 and the control loop mechanism 174 can be disengaged by the controller 142 when the first control loop 152 resumes operation to further adjusts the position of the exhalation valve 138 for flow rate and/or pressure regulation. Then, once a steady state condition is returned to by the exhalation valve 138, the second control loop 166 and the control loop mechanism 174 can be activated as require or desired. Generally, the first control loop 152 is used to perform pressure and/or flow rate control for the fluid circuit, while the second control loop 166 is used to perform position control of the exhalation valve 138 during vibration events.

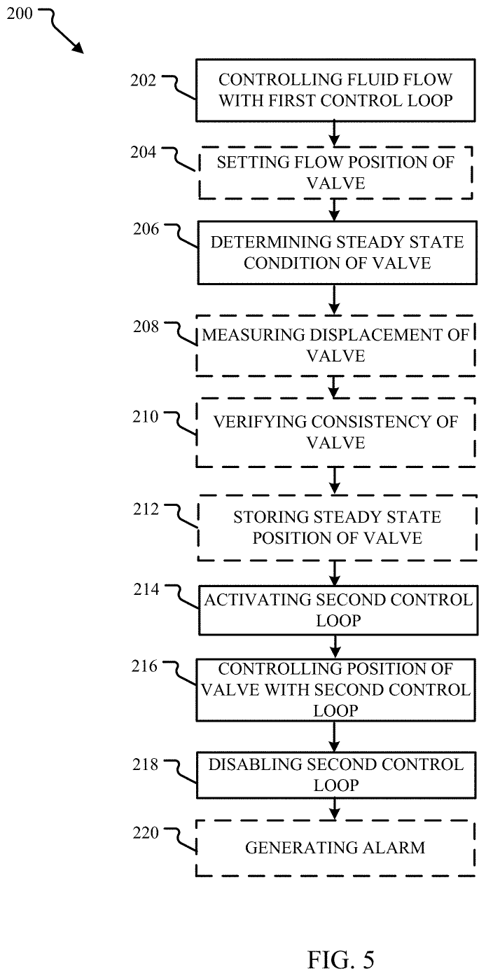

FIG. 5 is a flowchart illustrating a method 200 for stabilizing valve position on a ventilator. The example methods and operations can be implemented or performed by the systems and devices described herein (e.g., the ventilator 100). Elements of the methods illustrated by dashed borders may represent steps that are optional. The method 200 begins with controlling fluid flow through a valve with a first control loop (operation 202). The first control loop can be used to regulate fluid flow rate and/or pressure through the valve (e.g., an exhalation valve). In some examples, control with the first control loop (operation 202) includes setting a flow position of the valve based on a target fluid flow rate or pressure (operation 204). This flow position of the valve can be updated and revised due to feedback within the first control loop as required or desired.

The method 200 next flows to determining that the valve is in a steady state condition with respect to the first control loop (operation 206). The steady state condition relates to the position of the valve remaining unchanged, or relatively unchanged within a degree of error, over a period of time while in the first control loop. In some examples, determining the steady state condition can include measuring linear displacement of the valve over a plurality of sampling interval periods (operation 208) and verifying that the position of the valve remains substantially consistent over two or more sampling interval periods (operation 210). The measurement operations can be performed by the valve position sensor that is coupled to the valve. The positional measurements can be performed at a relatively fast sampling period compared to the flow rate or pressure measurement sampling period so as to provide a quick and responsive control loop. The displacement measurements can be an analog measurement that is converted to a digital value at an analog-to-digital converter (ADC) prior to being sent to the valve controller. In some examples, after determining that the valve is in a steady state condition (operation 206), a steady state position of the valve is stored (operation 212).

Once the steady state condition (operation 206) is determined, the method 200 includes activating a second control loop (operation 214) and then controlling a position of the valve with the second control loop based on a position sensor that measures linear displacement of the valve (operation 216). The second control loop can be used to stabilize valve position (e.g., due to vibratory forces) so as to maintain steady state valve position during the first control loop. This stabilization increases performance of the ventilator. In some examples, controlling the position of the valve with the second control loop (operation 216) is based at least partially on the stored steady state position of the valve relative to the measured linear displacement of the valve. As such, the second control loop is configured to maintain the steady state condition of the valve even when vibratory forces are induced into the system. This valve stabilization control loop can repeat itself as required or desired as a feedback loop so that the force applied to the valve is adjusted and based on the vibratory force being experienced by the valve so as to maintain the steady state position.

The method 200 then flows to disabling the second control loop when the first control loop is in operation (operation 218). By disabling the second control loop, the first control loop is able to modify or adjust the position of the valve so as to account for measured fluid flow rate and/or pressure changes and without the stabilizing operation of the second control loop. Then, once a steady state condition is returned to by the valve after positioning or repositioning, the stabilization control loop can be engaged and activated. As described herein, the first control loop is independent from the second control loop, and the second control loop operates at a smaller sampling interval period than the first control loop.

In another example, the method 200 can include generating an alarm on the ventilator when the second control loop is active (operation 220). The alarm may be an audio, a visual, or an audio and visual alarm that can alert the technologist to when high vibratory forces are present on the ventilator and based on operation of the stabilization control loop.

In the examples, controlling the position of the valve (e.g., in the first or the second control loops) is performed by electric current channeled to the valve. As such, a current, and thus a magnetic force, is determined by the controller to maintain the valve in the steady state position. This force corrects for the displacement of the valve from the vibratory forces so that flow rate and/or pressure changes based on valve position changes are reduced or prevented. As described herein, the magnetic force that generates positional change of the exhalation valve is at least partially based on the electric current that is channeled to the valve.

Although the present disclosure discusses the implementation of these techniques in the context of a ventilator capable of stabilizing valve position from vibratory forces, the techniques introduced above may be implemented for a variety of medical devices or devices utilizing sensors and control valves. A person of skill in the art will understand that the technology described in the context of a medical ventilator for human patients could be adapted for use with other systems such as ventilators for non-human patients, or general gas transport systems. Additionally, a person of ordinary skill in the art will understand that stabilizing valve position may be implemented in a variety of breathing circuit setups that may have a sensor and control valves. Further, while described primarily for use with an exhalation valve, the techniques described herein may also be used at the inhalation valve of the ventilator.

Those skilled in the art will recognize that the methods and systems of the present disclosure may be implemented in many manners, and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about,” “approximately,” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure.

What is claimed is:

1. A method of stabilizing valve position on a ventilator, the method comprising:
controlling fluid flow through a valve with a first control loop;
determining that the valve is in a steady state condition with respect to the first control loop;
activating a second control loop once the steady state condition is determined;
controlling a position of the valve with the second control loop based on a position sensor that measures linear displacement of the valve; and
disabling the second control loop when the first control loop is in operation, wherein the first control loop is independent from the second control loop, and wherein the second control loop operates at a smaller sampling interval period than the first control loop.

2. The method of claim 1, wherein controlling fluid flow through the valve with the first control loop comprises setting a flow position of the valve based on a target fluid flow rate or pressure.

3. The method of claim 1, further comprising after determining that the valve is in a steady state condition, storing a steady state position of the valve.

4. The method of claim 3, wherein controlling the position of the valve with the second control loop is based at least partially on the stored steady state position of the valve relative to the measured linear displacement of the valve.

5. The method of claim 1, wherein determining that the valve is in a steady state condition comprises:
measuring linear displacement of the valve over a plurality of sampling interval periods; and
verifying that the position of the valve remains substantially consistent over two or more sampling interval periods of the plurality of sampling interval periods.

6. The method of claim 1, further comprising generating an alarm on the ventilator when the second control loop is active.

7. The method of claim 1, wherein controlling the position of the valve is performed by electric current channeled to the valve.

8. A ventilator comprising:
a valve configured to regulate fluid flow therethrough;
a valve position sensor;
a processor; and
a memory storing computer executable instruction that when executed by the processor cause the ventilator to perform a set of operations that stabilizes the position of the valve comprising:
controlling the fluid flow through the valve with a first control loop;
determining that the valve is in a steady state condition with respect to the first control loop;
activating a second control loop once the steady state condition is determined;
controlling a position of the valve with the second control loop based on the valve position sensor that measures linear displacement of the valve; and
disabling the second control loop when the first control loop is in operation, wherein the first control loop is independent from the second control loop, and wherein the second control loop operates at a smaller sampling interval period than the first control loop.

9. The ventilator of claim 8, wherein the operation of controlling the fluid flow through the valve with the first control loop comprises setting a flow position of the valve based on a target fluid flow rate or pressure.

10. The ventilator of claim 8, wherein the set of operations further comprise after determining that the valve is in a steady state condition, storing a steady state position of the valve.

11. The ventilator of claim 10, wherein the operation of controlling the position of the valve with the second control loop is based at least partially on the stored steady state position of the valve relative to the measured linear displacement of the valve.

12. The ventilator of claim 8, wherein the operation of determining that the valve is in a steady state condition comprises:
measuring linear displacement of the valve over a plurality of sampling interval periods; and
verifying that the position of the valve remains substantially consistent over two or more sampling interval periods of the plurality of sampling interval periods.

13. The ventilator of claim 8, further comprising an audio, a visual, or an audio and visual alarm, and wherein the set of operations further comprise generating an alarm on the ventilator when the second control loop is active.

14. The ventilator of claim 8, wherein the operation of controlling the position of the valve is performed by electric current channeled to the valve.

15. A ventilator comprising:
a fluid flow circuit;
an exhalation valve coupled in fluidic communication with the fluid flow circuit and configured to at least partially control fluid flow through the fluid flow circuit;
a first sensor coupled to the fluid flow circuit and configured to measure flow rate, pressure, or flow rate and pressure of the fluid flow through the fluid flow circuit;
a second sensor coupled to the exhalation valve and configured to measure a position of the exhalation valve; and
a controller comprising a processor and memory coupled in communication with the exhalation valve, the first sensor, and the second sensor, wherein the controller is configured to control a position of the exhalation valve based on a target fluid flow rate or pressure in a first control loop having the first sensor and control the position of the exhalation valve based on measured linear displacement from the second sensor in a second control loop, wherein the first control loop is independent from the second control loop, and wherein the second control loop operates at a smaller sampling interval period than the first control loop.

16. The ventilator of claim 15, wherein the controller drives position of the exhalation valve by electric current.

17. The ventilator of claim 16, wherein the electric current is based at least partially on voltage calculated by the controller.

18. The ventilator of claim 15, wherein during operation of the first control loop, the second control loop is disabled by the controller.

19. The ventilator of claim 15, further comprising an audio, a visual, or an audio and visual alarm.

20. The ventilator of claim 15, further comprising at least one dampener configured to at least partially isolate the exhalation valve from vibratory forces.
